(19) United States
(12) Patent Application Publication
(10) Pub. No.: US 2017/0138930 A1
(43) Pub. Date: May 18, 2017

(54) NON-INVASIVE DIAGNOSTIC METHOD FOR THE EARLY DETECTION OF FETAL MALFORMATIONS

(71) Applicants: Jacopo TROISI, Montecorvino Pugliano (SA) (IT); Giovanni SCALA, Vico Equense (NA) (IT)

(72) Inventors: Jacopo Troisi, Montecorvino Pugliano (SA) (IT); Giovanni Scala, Vico Equense (NA) (IT); Maurizio Guida, Napoli (IT)

(21) Appl. No.: 15/310,197

(22) PCT Filed: May 7, 2015
(86) PCT No.: PCT/EP2015/060051

§ 371 Date: Nov. 10, 2016

(30) Foreign Application Priority Data
May 15, 2014 (IT) MI2014A000889

(51) Int. Cl. G01N 33/49; G06N 99/00; G06F 19/00

(52) U.S. Cl. CPC G01N 33/492; G06F 19/345; G06N 99/005; G01N 2800/385

(57) ABSTRACT

A non-invasive method for the early diagnosis of fetal malformations based on the metabolomics analysis of maternal blood is here described.

### INTRODUCTION

The present invention relates to a non-invasive method for early diagnosis of fetal malformations and, more specifically, to a non-invasive method for early diagnosis of fetal malformations based on the metabolomic analysis of maternal blood.

Fetal development defecs together reach a frequency of between 2 and 3% of all pregnancies (Hoyert D L, Mathews, T. J., Menacker F, et al. Annual summary of vital statistics: 2004. Pediatrics 2006; 117: 168-83), and are responsible for around 21% of perinatal and infant deaths (T. J. Mathews, M. S., and Marian f. MacDorman, Infant Mortality Statistics From the 2008 Period Linked Birth/Infant Death Data Set, National Vital Statistics Reports, 2012; 60 (5)), as well as for a significant number of disability cases and chronic diseases. For these reasons, the screening of fetal malformations is a common clinical practice in most developed countries. The most commonly used diagnostic methodology for this purpose is ultrasonography, which is a non-invasive method, safe for both the mother and the fetus. Its effectiveness in detecting fetal malformations, however, depends on the operator's experience and the quality of the equipment used, and is in any event decreased in particular clinical conditions such as oligohydramnios, maternal obesity, or complex fetal abnormalities.

The main limitation of such diagnostic methodology is the inability to detect birth defects before the second trimester of pregnancy. On the other hand, there are other diagnostic methods, such as chorionic villus sampling and amniocentesis, able to identify some of the defined disease malformations, already in the first trimester of pregnancy. However, these methods are useful only for some of defined congenital anomalies such as trisomy or other forms of cromosomopathies, and they are invasive, thus exposing both the mother and the fetus to a significant risk of serious complications.

Therefore the need for a non-invasive diagnostic method, capable of detecting fetal malformations in the first trimester of pregnancy, with good sensitivity and specificity is very felt.

The present invention relates to a non-invasive diagnostic method for early diagnosis of fetal malformations, based on the metabolomics analysis of maternal blood and on an integration of the obtained results by means of multivariate analysis that uses both models of PL-DA and OPLS-DA discriminant analysis and computer learning models as well (SVM and decision tree).

“Metabolomics” commonly defines the analysis of cellular processes through the study of the metabolic profile of small molecules of an organism. With “metabolomics analysis” inventors refer to the execution of a process aimed at the identification and the determination of the concentration of the greatest possible number of metabolites in a biological sample.

The term “metabolomics” commonly refers to the analysis of cellular processes by the metabolomics profile study of small molecules derived from an organism.

With the term “metabolomics profile” the inventors refer to the execution of a process aimed at the identification and the determination of the concentration of the greatest possible number of metabolites in a biological sample.

The term “metabolites” commonly refers to small molecules derived from the biological processes of anabolic or catabolic type of a cell or a set of cells. With the term “metabolites” the inventors refer to all the molecules with a molecular weight of less than 1000 Dalton, which are potentially identifiable and measurable within a biological sample.

To date, several thousands of metabolites in human serum have been identified and the application of metabolomics has allowed the development of biomarkers in many diseases such as schizophrenia (Kaddurah-Daouk R., Metabolic profiling of patients with schizophrenia, PLOS Med 2006; 8:e363), meningitis (Subramanian A. et al., Proton MR/CSF analysis and a new software as predictors for the differentiation of meningitis in children, NMR Biomed 2005; 18: 213-25) and colon cancer (C Denkert., et al., Metabolite profiling of human colon carcinoma—deregulation of TCA cuycle and amino acid turnover, Mol Cancer 2008; 7:1-15).

However, the use of metabolomics in obstetric has been so far limited to studies of preeclampsia (RO Bahado-Singh, R. Akolekar, R. Mandal et al., Metabolomics and first-trimester prediction of early-onset preeclampsia, Journal of Maternal-Fetal and Neonatal Medicine, vol. 25(10): 1840-7, 2012) restrictions on growth (RPHorgan, OF Broadhurst, SKWalsh et al., Metabolic profiling uncovers a phenotypic signature of small for gestational age in early pregnancy, Journal of Proteome Research, vol. 10(8): 3660-73, 2011); and studies using nuclear magnetic resonance (NMR). To date, studies conducted in gas chromatography coupled to mass spectrometry and chemometric techniques for the diagnosis of fetal malformations are not reported in literature.

### DESCRIPTION

The diagnostic method of the present invention is based on two phases. In a first stage samples from mothers with definitely malformed fetuses and samples from mothers with surely healthy fetuses are analyzed, and by means of these classification models are trained. This phase, defined as training phase, is designed to create and define the characteristics of the metabolic profile in the blood of the two groups. The expression “metabolic profile” refers to the specific pattern that the metabolites take in the patient blood, depending on their relative proportions.

In the second stage, the unknown samples are subjected to GCMS analysis, and the resulting chromatograms are classified according to the models previously trained, thus estimating the most probable class.

Therefore, the diagnostic process is not based on the measurement of the concentration of the individual metabolites, but the entire cluster of metabolites is considered as a biomarker; said metabolites allow for the insertion in two different classes in that they are present in different proportion in the two groups.

More in detail, the first phase is based on several sub-phases:

1. Extraction and derivatization of metabolites;
2. GCMS or GCxGCMS analysis;
3. Data array creation;
4. Structuring of the classification models.

The second phase involves the application of the first three sub-phases of the first phase to the unknown sample and the attribution of the most likely class of membership on the basis of the question of the classification model formulated in the first phase.

The method of the present invention has the advantage that it can be used already in the first trimester of gestation.

### Extraction and Derivatization of the Metabolites

50 L of haemolysed blood are transferred into 2 mL Eppendorf tubes and 20 μL of a solution of 1 g/L of Ribitol and 500 μL of methanol are added. The solution is mixed in a vortex for 30 seconds. After heating for 15 minutes at 70° C., the samples are centrifuged at 10,000 rpm for 10 minutes at 20° C. An aliquot of 200 μL of the supernatant is collected and transferred to new 2 mL Eppendorf tubes and added with 200 μL of water and 100 μL of chloroform, mixed in a vortex for 30 seconds and centrifuged at 4,000 rpm for 15 minutes at 20° C. An aliquot of 200 μL of the supernatant is again collected and transferred into 0.2 mL glass vials, dried under nitrogen flow, and then added with 50 μL of methoxylamine hydrochloride 20 mg/mL in pyridine and the reaction is conducted in the dark at 20° C. for 16 hours. At the end, 50 μL of N,O-bis(trimethylsilyl)trifluoroacetamide (BSTFA) with 1% trimethylchlorosilane (TMCS) are added to each vial and the silanization reaction is conducted at 70° C. for 16 hours.

To obtain a separation between the metabolites useful to the purposes of this invention, it is possible to operate in one-dimensional gas chromatography and in two-dimensional gas chromatography as well. The best resolving power of the two-dimensional technique potentially offers a better accuracy in classification, but it is also possible to operate with one-dimensional gas chromatography—which is the most commonly known—so as shown in the “Experimental evidence of the operation of the invention”.

### MDGCMS Analysis

For the two-dimensional gas chromatography a primary column (placed in the first oven) of the type SLB-5 ms 20.0 mx 0.18 mm ID with 0.10 μm of film thickness [sylphenilene polymer, which is virtually equivalent in polarity to poly(5% diphenyl/95% methylsiloxane)] (Supelco) is used, and it is connected to the position 1 of 7 ports interface (SGE). A BPX-50 5.0 m×0.25 mm ID with 0.25 μm of the film thickness is connected to the position 7 of the interface. A BPX-50 1.5 m×0.25 mm ID, 0.25 μm is fixed at the position 6 and connected to a flame ionization detector (FID) put at 320° C., while the analytical column of 5.0 m (chemically identical to the one connected to the FID) is connected to the qMS system. The column connected to the FID is used to reduce the flow in the second dimension and to verify that a unrepresentative compound is not the result of a random fluctuation of the chromatography. A 40 μL external capillary (20 cm×0.71 mm OD×0.51 mm ID made of stainless steel) is used to connect the ports 3 and 4 of SGE interface. The temperature program is the same for the two ovens: 80° C. for 1 minute and then heating up to 320° C. at 3° C./minute and held for 1 minute. The initial helium pressure (constant linear velocity) is fixed at 129.6 kPa. The initial auxiliary helium pressure APC (advanced control pressure), which is also operating in conditions of constant linear velocity, is set at 90.4 kPa, the injection volume to 1 μL with a split ratio: 1:10. The modulation period is set at 4.1 s (accumulation period of 4.0 seconds, the injection period of 0.1 second). The conditions of the mass spectrometer quadrupole are: ionization mode: electron impact (70 eV), mass range: 40-800 m/z, scanning speed: 10,000 amu/second.

### GCMS Analysis

For the one-dimensional gas chromatography a column of type ZB5-ms 60.0 m×0.25 mm ID×0.25 μm [sylphenilene polimer, virtually equivalent in polarity to poly(5% diphenyl/95% methylsiloxane)] (Phenomenex) is used.

The temperature program of GC provides 80° C. for 1 minute and then heating up to 300° C. at 3° C./minute and 1.67 minutes of hold time. The initial helium pressure (constant linear velocity) is fixed at 129.6 kPa. The injection volume to 1 μL with a split ratio: 1:2. The conditions of the quadrupole mass spectrometer are: ionization mode: electron impact (70 eV), mass range: 40-800 m/z, scan speed: 10,000 amu/second.

### Data Array Creation

The gas chromatograms obtained in SCAN mode are integrated in order to identify all the peaks having an area greater than 10 times the background noise of the gas chromatographic plot. Each peak must be identified on the basis of one quantization m/z signal and at least on 2 qualification m/z signals. In consequence of the integration, the quantification is carried out with the method of the normalized percentages areas, the peak of Ribitol is used as a reference for the quantitative analysis and for the centering of the retention times. The results obtained by this quantization (percentage areas normalized) are transferred to a matrix in which each sample represents a line and the columns are represented by various metabolites, uniquely identified by means of their gas chromatographic retention time.

The first column of the matrix is used to define the class of the sample. In the simplest scenario only two classes “normal fetus” and “malformed fetus” can be envisaged; evidence of the invention based on this dichotomous classification are shown by the inventors in the “Experimental evidence of the operation of the invention”, but they consider that it is possible to imagine more complex classification scenarios where specific malformation classes can be separated, by placing a sufficient number of observations.

### Structuring of Classification Models

Different classification models are suitable for the purpose of the present invention; in particular, the performance of PLS-DA, OPLS-DA, SVM and decision tree models have been positively evaluated.

### PLS-DA

PLS is a supervised method that uses multivariate regression techniques to extract the information that may provide for the membership of a particular class (Y) by linear combinations of the original variables (X). The PLS regression is performed using the PLSR function provided by the pls package of the R language (Ron Wehrens and Bjorn Helge-Mevik. Pls: Partial Least Squares Regression (PLSR) and Principal Component Regression (PCR), 2007, R package version 2.1-0). Classification and cross-validation are performed using the corresponding wrapper function by the caret package (Max Kuhn. Contributions from Jed Wing and Steve Weston and Andre Williams. caret: Classification and Regression Training, 2008, R package version 3.45). In order to evaluate the effectiveness in classes discrimination, a permutation test is performed. In each permutation, a PLS-DA model is built from the data (X) and the commuted class labels (Y) by using the optimal number of components determined by cross validation for the model based on the assignment of the original classes. Two types of statistical tests are performed to measure the discrimination power between classes. The first is based on the prediction accuracy in the training phase of the model. The second is based on the separation distance according to the ratio between the sum of the quadratic distances within the classes and among the classes (B/W-ratio).

### OPLS-DA

Orthogonal Partial Least Squares-Discriminant Analysis (OPLS-DA) is an important development of the technique PLS-DA that has been proposed to manage the variation of the orthogonally class in the data matrix. OPLS-DA increases the classification performances of the PLS-DA models. The performances of classification are estimated on the basis of “k-fold cross validation” by dividing the array of data in k random subsets. For each calculation cycle, one of the subsets of k is kept aside as a test set and the remaining k−1 subsets act as trainers. Each of the k subsets is used one time as a test set, generating k precision values. The accuracy of the classification is calculated as the average of the accuracy rates in k subsets. The model is subjected to cross validation with the method leave one out cross validation (LOOCV), in order to be validated. To perform the classification is chosen the kernel parameter, which corresponds to the maximum precision of the cross validation. The data matrix is scaled to the mean and the unit variance, before being submitted to the division into k subsets. In other words, the average and the standard deviation of the training data are used to indicate the center and to scale the test data. Once trained, the model is used to check whether the data have generated an “overfitting”. To do this, a validation set with known class labels is created and it is thus verified whether it gives an accuracy rate comparable to that of the training data. Another method is a plot validation R²/Q² which helps to assess the risk that the current model is spurious, ie, the model fits well only to subsets set but does not predict Y just as well for the new observations. The value of R² is the percentage variation of the training set that can be explained by the model. The value of Q² is a measure of cross validated R². This validation compares the goodness of fit of the original model with the goodness of fit of the different models based on the data in which the order of the observations Y is permuted randomly, while the matrix is kept intact. The criteria for the validity of the model are the following:
1. All the Q² values on the permuted data set must be lower than the value Q² value, estimated on the current data set. If this is not checked, it means that the model is overfitted.
2. The regression line (the line joining the point Q² real to the centroid of the cluster of values permuted Q²) has a negative value of the y-axis intercept.

### SVM

Support Vector Machines (SVMs) are machine learning supervised techniques relatively new for classification uses. The SVMs were proposed for the first time in 1982 by Vapnik (Vapnik, V. Estimation of Dependences Based on Empirical Data, Springer Verlag: New York, 1982). The basic principle of SVMs, which are essentially binary classifiers, is the following: given a set of data with two classes, a linear classifier is constructed in the form of a hyperplane, which has the maximum margin in the simultaneous minimization of the empirical classification error and the maximization of the geometric margin. In the case of data sets that are not linearly separable, the original data are mapped into a higher dimensional feature space and a linear classifier is built in this new space (this is known as the “kernel”), which is equivalent to the construction of a linear classifier in the space of the original input. This mapping is implicitly given by the kernel function.

Given a set of training data Xᵢ [symbol] Rⁿ, i=1, . . . , m where each Xᵢ falls into one of two categories yᵢ [symbol] {−1,1}, SVM determines the hyperplane whose parameters are given by (w, b) so as obtained from the solution of the following convex optimization problem:

$${min}_{w , b , ε} \frac{1}{2} {w}^{t} w + c {\sum }_{i = 1}^{m} {ε}_{i}$$

which is subjected to the following conditions

$${y}_{i} ( {w}^{t} {x}_{i} + b ) \geq 1 - {ε}_{i}$$

$${ε}_{i} \geq 0$$

where c is the regularization parameter, which is a compromise between the learning accuracy and the term prediction, and ε is a measure of the number of classification errors. The inclusion of the term regularization reduces the problem of overfitting.

### Decision Trees

Decision trees build classification models based on recursive partitioning of data. Typically, an algorithm of the decision tree begins with the entire set of data, the data are divided into two or more subgroups based on the values of one or more attributes, and then each subset is repeatedly divided in smaller subsets until the size of each subset reaches an appropriate level. The entire modeling process can be represented in a tree structure, and the generated model can be summarized as a set of rules “if-then”. Decision trees are easy to interpret, computationally undemanding, and able to cope with noisy data. Most of the decision trees tackles the classification problems, which is also the object of this invention. In this context, the technique is also referred to as classification tree. In the representation with the tree structure, a node represents a set of data, and the entire set of data is represented as a node at the root.

### Experimental Evidence of the Invention Functioning

The diagnostic method of the present invention has been developed starting from the metabolomics analysis, carried out on blood samples collected from pregnant women with diagnosis of fetal malformation and from control pregnant women, with the clinical certainty of absence of fetal malformation pathologies.

### Sample Collection

The samples were collected from 100 healthy pregnant women, who have undergone abortion following the diagnosis of fetal malformation, and have voluntarily donated blood samples. Blood samples were taken immediately before the termination of pregnancy using BD Vacutainer® tubes, and frozen at −30° C. until analysis. The suspected diagnosis of fetal malformation due to amniocentesis or ultrasound examination was confirmed by autopic post explant fetal examination. Each blood sample was associated with an equivalent control sample taken from a person to the same week of gestation and with similar personal, physical and social characteristics (weight, height, body mass index, age, marital status, economic status, etc.).

### Extraction and Derivatization of the Metabolites

The extraction and derivatization of the samples were conducted in accordance with the provisions in the DESCRIPTION paragraph.

### Chromatographic Determinations

The GCMS analysis and GCxGCMS were carried out with Shimadzu instruments according to the information given in the DESCRIPTION paragraph.

### Statistical Analysis

The multivariate statistical analysis of the data (PLS-DA and OPLS-DA) and the machine learning (SVM and decision tree) were performed on a chromatogram, normalized and corrected (based on the peak area of Ribitol) using SIMPCA-P 13.0 (Umetrics), RapidMiner 5.3 (Rapid-I) and R (Foundation for Statistial Computing, Vienna). The values have been centered on the mean and the variance was normalized.

### Results

The results were obtained from 100 cases of fetal malformation (FM) and from 100 controls. The demographic and clinical characteristics of the cases of FM and controls are shown in Table 1, whereas the investigated malformations are listed in Table 2.

TABLE 1
characteristics of the study population

| Parameter | Fetal Malformation | Controls | P Value |
|---|---|---|---|
| Number of cases | 100 | 100 | — |
| Maternal age (years) | 34 ± 6.9 | 32 ± 6.1 | NS |
| Gestational age (weeks) | 19.2 ± 2.6 | 18.9 ± 2.2 | NS |
| Equality | 1.8 ± 0.9 | 1.7 ± 1.1 | NS |

TABLE 2
malformations investigated

| | | Number |
|---|---|---|
| | | of |
| Type | Malformation | cases |
| Abnormalities | Acrania, anencephaly, agenesis of the | 26 |
| of the central | corpus callosum, hydrocephalus, cystic | |
| nervous system | hygroma, myelomeningocele, spina | |
| and peripheral | bifida, spinal muscular atrophy, | |
| | leukodystrophy Krabbe disease | |
| | Werding-Hoffmann, Dandy Walker | |
| | syndrome | |
| Chromosomal | Trisomy 21, trisomy 18, trisomy 13, | 46 |
| abnormalities | balanced translocation, unbalanced | |
| | translocation, Turner, X0/XY | |
| Polymalformations | Twins polymalformation, poly- | 9 |
| | malformation, Ellis van Creveld | |
| | syndrome | |
| Cardiac | Fallot tetralogy, complex cardiac | 4 |
| abnormalities | malformations, heart disease | |
| Haematological | Fetal hydrops, major thalassemia | 4 |
| abnormalities | | |
| Abnormalities | Budd Chiari syndrome, diaphragmatic | 3 |
| of the digestive | hernia, syndrome Bochdalek | |
| system | | |
| Anomalies of the | Renal agenesis renal dysplasia, Potter | 3 |
| urogenital system | syndrome | |
| Bone and skeletal | Imperfecta osteogenesis | 2 |
| system abnormalities | | |
| Other | Cystic fibrosis, non-immune fetal | 2 |
| | hydrops | |

In a TIC chromatogram are normally recognized more than 150 signals in a single sample and some of these peaks were not further investigated because they were not found correspondingly in other samples, because of in too low concentration or because of poor spectral quality in order to be confirmed as metabolites. A total of 116 endogenous metabolites such as amino acids, organic acids, carbohydrates, fatty acids and steroids were detected. For the identification of the peak, the linear retention index (LRI) was used by placing as tolerance a difference between the tabulated data and those identified of maximum 10, while the minimum of compatibility for the search in the NIST library was placed at 85% minimum. The peak areas were normalized and corrected to Ribitol signal. The results were summarized in a matrix file, separated by comma (CSV) and loaded into an appropriate software for statistical processing.

For the metabolic profile, the model OPLS-DA showed satisfactory predictive and modeling capabilities by using a predictive component and three orthogonal components (R²Ycum=0.971, Q²cum=0.372). The other classification models showed good classification capacity (although lower than ‘OPLS-DA). Several approaches are possible for the definitive allocation of the class of the unknown sample. It is possible to use the response of a single model or to integrate the responses of individual models in a more complex decision algorithm.

1) A method for the early diagnosis of fetal malformations based on the metabolomics analysis of the maternal blood, said method comprising
a first phase comprising analyzing blood samples collected from mothers with malformed fetuses and from mothers with healthy fetuses to train the classification models and
a second phase comprising analyzing GCMS of blood samples collected from pregnant women and assigning a class membership on the basis of the classification model established in the first phase.

2) A method according to claim 1 wherein the first phase comprises:
1. extracting and derivatizing metabolites;
2. performing GCMS or GCxGCMS analysis;
3. creating a data matrix; and
4. Structuring classification models.

3) A method according to claim 1 wherein the second phase comprises:
1. extracting and derivatizing metabolites;
2. performing GCMS or GCxGCMS analysis;
3. creating a data matrix;
4. assigning class membership.

4) A method according to claim 1 for the diagnosis of fetal malformation in the first trimester of pregnancy.
